(19) United States
(12) Patent Application Publication
(10) Pub. No.: US 2011/0305658 A1
(43) Pub. Date: Dec. 15, 2011

(54) LENGTHENING MASCARA COMPOSITION

(75) Inventors: Hy Si Bui, Piscataway, NJ (US); Mohamed Kanji, Edison, NJ (US); Anita Chon Tong, Garwood, NJ (US); Bruno Bavouzet, Hoboken, NJ (US)

(73) Assignee: L'OREAL S.A., Paris (FR)

(21) Appl. No.: 13/133,688

(22) PCT Filed: Dec. 11, 2009
(86) PCT No.: PCT/US09/67713

§ 371 Date: Aug. 8, 2011

Related U.S. Application Data
(60) Provisional application No. 61/121,579, filed on Dec. 11, 2008.
(60) Provisional application No. 61/121,576, filed on Dec. 11, 2008.

(51) Int. Cl. A61K 8/92; A61Q 1/10

(52) U.S. Cl. 424/70.7

(57) ABSTRACT

The present invention is directed to a lengthening and smudge proof mascara composition for making up eye lashes having: (a) at least one alkyl ethoxylated polymer wax; (b) at least one oil soluble polar modified polymer; (c) at least one oil soluble high carbon polar modified polymer; (d) water; (e) at least one non-volatile oil capable of solubilizing the polar modified polymers; (f) optionally, at least one volatile solvent; and (g) optionally, at least one colorant, and wherein the composition does not require fibers to be dispersed therein.

### FIELD OF THE INVENTION

The present invention generally relates to a novel mascara composition and method of making-up eye lashes. More particularly, the present invention relates to a self-emulsifying, lengthening and smudge proof mascara composition which, until now, could only be achieved through the use of fibers.

### BACKGROUND OF THE INVENTION

It is well known in the industry that the primary way of making a mascara composition capable of lengthening eyelashes is to disperse fibers therein. The problem with fiber-based lengthening mascaras is that the fibers have a tendency to attach themselves very unevenly, and to only a few eye lashes, thereby yielding sparse and uneven lengthening.

Also, when formulating water-containing mascara compositions, surfactants/emulsifiers are conventionally used in order to form a stable emulsion. This, in turn, adds to the overall cost of the product.

Therefore, it is an object of the present invention to provide a stable, self-emulsifying, lengthening and smudge proof mascara composition, in oil-in-water/water-in-oil emulsion form, having improved eye lash lengthening properties which does not require fibers to be dispersed therein.

The present invention also generally relates to a novel mascara composition and method of making-up eye lashes.

### BRIEF SUMMARY OF THE INVENTION

In one embodiment of the present invention, a composition comprises: (a) at least one alkyl ethoxylated polymer wax; (b) at least one oil soluble polar modified polymer; (c) at least one oil soluble high carbon polar modified polymer; (d) water; (e) at least one non-volatile oil capable of solubilizing the polar modified polymers; (f) optionally, at least one volatile solvent; and (g) optionally, at least one colorant, and wherein the composition does not require fibers to be dispersed therein.

In another embodiment of the present invention, a composition comprises: (a) at least one alkyl ethoxylated polymer wax; (b) at least one oil soluble polar modified polymer; (c) at least one oil soluble high carbon polar modified polymer; (d) water; (e) at least one non-volatile oil capable of solubilizing the polar modified polymers; (f) optionally, at least one volatile solvent; (g) at least one high molecular weight glycol; and(h) optionally, at least one colorant, and wherein the composition does not require fibers to be dispersed therein.

Another aspect of the present invention is directed to a method of making up a keratinous substrate involving applying the above-disclosed compositions onto the substrate.

It has been surprisingly and unexpectedly discovered that the above-disclosed composition, when applied onto eye lashes, can lengthen the appearance of eye lashes by creating an extension thereon, without having to disperse fibers in the composition.

It has also been surprisingly discovered that the composition of the present invention forms a stable, smudge proof, long wear emulsion, having a unique texture and feel, without the need for having to employ a surfactant/emulsifier to form the emulsion. In addition, the composition possesses long wear properties without the need for having to employ conventional latex or other film forming polymers.

### BRIEF DESCRIPTION OF THE DRAWINGS

FIG. 1 depicts the rheology of mascara example 4 as compared with Maybelline Sky High Curve Washable Mascara.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

“Film former” or “film forming agent” or “film forming resin” as used herein means a polymer which, after dissolution in at least one solvent (such as, for example, water and organic solvents), leaves a film on the substrate to which it is applied, for example, once the at least one solvent evaporates, absorbs and/or dissipates on the substrate.

“Tackiness”, as used herein, refers to the adhesion between two substances. For example, the more tackiness there is between two substances, the more adhesion there is between the substances.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as phase separation if the composition is in the form of an emulsion, bending or leaning if the composition is in stick form, melting, or syneresis (or sweating). The stability is further tested by repeating the 8-week test at 37° C., 40° C., 45° C., 50° C., and under freeze-thaw conditions. A composition is considered to lack stability if in any of these tests an abnormality that impedes functioning of the composition is observed. The skilled artisan will readily recognize an abnormality that impedes functioning of a composition based on the intended application.

“Volatile”, as used herein, means having a flash point of less than about 100° C.

“Non-volatile”, as used herein, means having a flash point of greater than about 100° C.

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number.

“Smudge proof” as used herein refers to the ability to repel sebum and permanence with respect to squalene. Smudge proof properties may be evaluated by any method known in the art for evaluating such properties. For example, a mascara composition may be applied to false eyelashes, which may then be placed in squalene for a certain amount of time, such as, for example, 20 minutes. Upon expiration of the pre-ascertained amount of time, the false eyelashes may be removed from the squalene and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in squalene for a certain amount of time. The amount of composition remaining on the skin after the pre-ascertained amount of time may then be evaluated and compared. For example, a composition may be smudge proof if a majority of the product is left on the wearer, e.g., eyelashes, skin, etc. In a preferred embodiment of the present invention, little or no composition is transferred from the wearer.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

Alkyl Ethoxylated Polymer Waxes

The compositions of the present invention comprise at least one alkyl ethoxylated polymer wax that may be selected from di-alkyl, tri-alkyl- and combinations of di-alkyl and tri-alkyl substituted alkyl ethoxylated polymer waxes. Alternatively mono-alkyl, di-alkyl, tri-alkyl, tetra-alkyl and all combinations thereof substituted alkyl ethoxylated polymer waxes. The alkyl group can be saturated or unsaturated, branched or linear and contain a number of carbon atoms from about 12 carbon atoms to about 50 carbon atoms.

The alkyl substitution of the alkyl ethoxylated polymer wax includes mono-alkyl, di-alkyl, tri-alkyl and tetra-alkyl substitution of the polymer wax and combinations thereof. Examples of the polymer waxes that are mono alkyl substituted include: Steareth-100 available as Brij 700 from Uniqema Inc., Pareth alcohols available as Performathox 450, 480 and 490 available from New Phase Technologies, Inc. The di-alkyl substituted polymer waxes include PEG 120 methyl glucose dioleate available as Glutamate DOE-120 and Glucamate DOE-120 both from Chemron Corporation. The tri-alkyl substituted polymer waxes include PEG 120 methyl glucose trioleate available as Glucamate LT from Chemron Corporation. The tetra-alkyl substituted polymer waxes include PEG 150 pentaerythrityl tetrastearate available as Crothix from Croda Corporation.

In the present invention, preferred alky ethoxylated polymer waxes include ethoxylated C₂₀₋₅₀ fatty alcohols having an average molecular weight of the alcohol chain of from about 450 to 550 and an average degree of ethoxylation of from about 2.5 to 95. These alkyl ethoxylated waxes have a melting point ranging from 70 to 100° C. The most preferred waxes are Pareth-10 alcohol which is a mixture of C₂₀₋₄₀ fatty alcohols having an average molecular weight of about 450 and average degree of ethoxylation of about 10, commercially available as Performathox 450, and Pareth-40 alcohol, which is a mixture of C₂₀₋₄₀ fatty alcohols having an average molecular weight of about 450 and an average degree of ethoxylation of about 42, commercially available as Performathox 480, both from New Phase Technologies, Inc.

Preferably, the alkyl ethoxylated polymer wax(es) represent from about 3% to about 30% by weight of the total weight of the composition, more preferably from about 5% to about 20% by weight of the total weight of the composition, and most preferably from about 7% to about 15% by weight of the total composition, including all ranges and subranges therebetween.

Oil-Soluble Polar Modified Polymer

According to the present invention, compositions comprising at least one oil-soluble polar modified polymer are provided. “Polar modified polymer” as used herein refers to a hydrophobic homopolymer or copolymer which has been modified with hydrophilic unit(s). “Oil-soluble” as used herein means that the polar modified polymer is soluble in oil.

Suitable monomers for the hydrophobic homopolymers and/or copolymers include, but are not limited to, cyclic, linear or branched, substituted or unsubstituted, C2-C20 compounds such as, for example, styrene, ethylene, propylene, isopropylene, butylene, isobutylene, pentene, isopentene, isoprene, hexene, isohexene, decene, isodecene, and octadecene, including all ranges and subranges therebetween. Preferably, the monomers are C2-C8 compounds, more preferably C2-C6 compounds, and most preferably C2-C4 compounds such as ethylene, propylene and butylene.

Suitable hydrophilic unit(s) include, but are not limited to, maleic anhydride, acrylates, alkyl acrylates such as, for example, methyl acrylate, ethyl acrylate, propyl acrylate, and butyl acrylate, and polyvinylpyrrolidone (PVP).

According to the present invention, the polar modified polymer is oil-soluble: that is, the polymer does not contain a sufficient amount of hydrophilic unit(s) to render the entire polymer water-soluble or oil-insoluble. According to preferred embodiments, the polar modified polymer contains the same amount of hydrophobic monomer as hydrophilic unit (1:1 ratio) or more hydrophobic monomer than hydrophilic unit. According to particularly preferred embodiments, the polar modified polymer contains 50% or less hydrophilic unit(s) (based on weight of the polymer), 40% or less hydrophilic unit(s), 30% or less hydrophilic unit(s), 20% or less hydrophilic unit(s), 10% or less hydrophilic unit(s), 5% or less hydrophilic unit(s), 4% or less hydrophilic unit(s), or 3% or less hydrophilic unit(s).

Preferably, the polar modified polymer has from about 0.5% to about 10% hydrophilic units, more preferably from about 1% to about 8% hydrophilic units by weight with respect to the weight of the polymer, including all ranges and subranges therebetween. Particularly preferred hydrophilically modified polymers are ethylene and/or propylene homopolymers and copolymers which have been modified with maleic anhydride units.

According to preferred embodiments of the present invention, the polar modified polymer is a wax. According to particularly preferred embodiments, the polar modified wax is made via metallocene catalysis, and includes polar groups or units as well as a hydrophobic backbone. Suitable modified waxes include those disclosed in U.S. patent application publication no. 20070031361, the entire contents of which is hereby incorporated by reference. Particularly preferred polar modified waxes are C2-C3 polar modified waxes.

In accordance with preferred embodiments of the present invention, the polar modified wax is based upon a homopolymer and/or copolymer wax of hydrophobic monomers and has a weight-average molecular weight Mw of less than or equal to 25 000 g/mol, preferably of 1000 to 22 000 g/mol and particularly preferably of 4000 to 20,000 g/mol, a number-average molecular weight Mn of less than or equal to 15 000 g/mol, preferably of 500 to 12 000 g/mol and particularly preferably of 1000 to 5000 g/mol, a molar mass distribution Mw/Mn in the range from 1.5 to 10, preferably from 1.5 to 5, particularly preferably from 1.5 to 3 and especially preferably from 2 to 2.5, which have been obtained by metallocene catalysis. Also, the polar modified wax preferably has a melting point above 75° C., more preferably above 90° C. such as, for example, a melting point between 90° C. and 160° C., preferably between 100° C. and 150° C., including all ranges and subranges therebetween.

In the case of a copolymer wax, it is preferable to have, based on the total weight of the copolymer backbone, 0.1 to 30% by weight of structural units originating from the one monomer and 70.0 to 99.9% by weight of structural units originating from the other monomer. Such homopolymer and copolymer waxes can be made, for example, by the process described in EP 571 882, the entire contents of which is hereby incorporated by reference, using the metallocene catalysts specified therein. Suitable preparation processes include, for example, suspension polymerization, solution polymerization and gas-phase polymerization of olefins in the presence of metallocene catalysts, with polymerization in the monomers also being possible.

Polar modified waxes can be produced in a known manner from the hompopolymers and copolymers described above by oxidation with oxygen-containing gases, for example air, or by graft reaction with polar monomers, for example maleic acid or acrylic acid or derivatives of these acids. The polar modification of metallocene polyolefin waxes by oxidation with air is described, for example, in EP 0 890 583 A1, and the modification by grafting is described, for example, in U.S. Pat. No. 5,998,547, the entire contents of both of which are hereby incorporated by reference in their entirety.

Acceptable polar modified waxes include, but are not limited to, homopolymers and/or copolymers of ethylene and/or propylene groups which have been modified with hydrophilic units such as, for example, maleic anhydride, acrylate, methacrylate, polyvinylpyrrolidone (PVP), etc. Preferably, the C2-C3 wax has from about 0.5% to about 10% hydrophilic units, more preferably from about 1% to about 8% hydrophilic units by weight with respect to the weight of the wax, including all ranges and subranges therebetween. Particularly preferred hydrophilically modified waxes are ethylene and/or propylene homopolymers and copolymers which have been modified with maleic anhydride units.

Particularly preferred C2-C3 polar modified waxes for use in the present invention are polypropylene and/or polyethylene-maleic anhydride modified waxes (“PEMA,” “PPMA.” “PEPPMA”) commercially available from Clariant under the trade name LICOCARE or LICOCENE, Specific examples of such waxes include products marketed by Clariant under the LicoCare name having designations such as PP207.

Other suitable polar modified polymers include, but are not limited to A-C 573 A (ETHYLENE-MALEIC ANHYDRIDE COPOLYMER; Drop Point, Mettler: 106° C.) from Honeywell, A-C 596 A (PROPYLENE-MALEIC ANHYDRIDE COPOLYMER; Drop Point, Mettler: 143° C.) from Honeywell, A-C 597 (PROPYLENE-MALEIC ANHYDRIDE COPOLYMER; Drop Point, Mettler: 141° C.) from Honeywell, ZeMac® copolymers (from VERTELLUS) which are 1:1 copolymers of ethylene and maleic anhydride, polyisobutylene-maleic anhydride sold under the trade name ISOBAM (from Kuraray), polyisoprene-graft-maleic anhydride sold by Sigma Aldrich, poly(maleic anhydride-octadecene) sold by Chevron Philips Chemcial Co., poly(ethylene-co-butyl acrylate-co-maleic anhydride) sold under the trade name of Lotader (e.g. 2210, 3210, 4210, and 3410 grades) by Arkema, copolymers in which the butyl acrylate is replaced by other alkyl acrylates (including methyl acrylate [grades 3430, 4404, and 4503] and ethyl acrylate [grades 6200, 8200, 3300, TX 8030, 7500, 5500, 4700, and 4720) also sold by Arkema under the Lotader name, and isobutylene maleic anhydride copolymer sold under the name ACO-5013 by ISP.

According to other embodiments of the present invention, the polar modified polymer is not a wax. In accordance with these embodiments of the present invention, the polar modified polymer is based upon a homopolymer and/or copolymer of hydrophobic monomer(s) and has a weight-average molecular weight Mw of less than or equal to 1,000,000 g/mol, preferably of 1000 to 250,000 g/mol and particularly preferably of 5,000 to 50,000 g/mol, including all ranges and subranges therebetween.

In accordance with these embodiments, the polar modified polymer can be of any form typically associated with polymers such as, for example, block copolymer, a grafted copolymer or an alternating copolymer. For example, the polar modified polymer can contain a hydrophobic backbone (such as polypropylene and/or polyethylene) onto which hydrophilic groups (such as maleic anhydride) have been attached by any means including, for example, grafting. The attached groups can have any orientation (for example, atactic, isotactic or syndiotactic along the backbone).

Preferably, the oil soluble polar modified polymer(s) represent from about 1% to about 30% of the total weight of the composition, more preferably from about 2% to about 20% of the total weight of the composition, and most preferably from about 5% to about 15%, including all ranges and subranges therebetween.

Oil-Soluble High Carbon Polar Modified Polymer

According to the present invention, compositions comprising at least one oil-soluble high carbon polar modified polymer are provided. “Polar modified polymer” as used herein refers to a hydrophobic homopolymer or copolymer which has been modified with hydrophilic unit(s). “Oil-soluble” as used herein means that the polar modified polymer is soluble in oil. “High carbon” means more than 20 carbon atoms.

Suitable monomers for the hydrophobic homopolymers and/or copolymers include, but are not limited to, cyclic, linear or branched, substituted or unsubstituted, C22-C40 compounds such as, C22-C28 compounds, C24-C26 compounds, C26-C28 compounds, and C30-C38 compounds, including all ranges and subranges therebetween. Preferably, the monomers are C24-26 compounds, C26-C28 compounds or C30-C38 compounds.

Suitable hydrophilic unit(s) include, but are not limited to, maleic anhydride, acrylates, alkyl acrylates such as, for example, methyl acrylate, ethyl acrylate, propyl acrylate, and butyl acrylate, and polyvinylpyrrolidone (PVP).

According to preferred embodiments, the oil-soluble high carbon polar modified polymer is a wax. Also preferably, the oil-soluble high carbon polar modified polymer wax has one or more of the following properties:

a weight-average molecular weight Mw of less than or equal to 30 000 g/mol, preferably of 500 to 10 000 g/mol and particularly preferably of 1000 to 5,000 g/mol, including all ranges and subranges therebetween;

a number-average molecular weight Mn of less than or equal to 15 000 g/mol, preferably of 500 to 12 000 g/mol and particularly preferably of 1000 to 5000 g/mol, including all ranges and subranges therebetween;

a molar mass distribution Mw/Mn in the range from 1.5 to 10, preferably from 1.5 to 5, particularly preferably from 1.5 to 3 and especially preferably from 2 to 2.5, including all ranges and subranges therebetween; and/or

a crystallinity of 8% to 60%, preferably 9% to 40%, and more preferably 10% to 30%, including all ranges and subranges therebetween, as determined by differential scanning calorimetry.

According to preferred embodiments relating to a copolymer wax, it is preferable to have, based on the total weight of the copolymer backbone, 0.1 to 30% by weight of structural units originating from the one monomer and 70.0 to 99.9% by weight of structural units originating from the other monomer.

Waxes of the present invention can be based upon homopolymers or copolymers made, for example, by the process described in EP 571 882, the entire contents of which is hereby incorporated by reference. Suitable preparation processes include, for example, suspension polymerization, solution polymerization and gas-phase polymerization of olefins in the presence of catalysts, with polymerization in the monomers also being possible.

Oil-soluble high carbon polar modified polymer wax can be produced in a known manner from the hompopolymers and copolymers described above by oxidation with oxygen-containing gases, for example air, or by graft reaction with polar monomers, for example maleic acid or acrylic acid or derivatives of these acids. The polar modification of polyolefin waxes by oxidation with air is described, for example, in EP 0 890 583 A1, and the modification by grafting is described, for example, in U.S. Pat. No. 5,998,547, the entire contents of both of which are hereby incorporated by reference in their entirety.

Acceptable oil-soluble high carbon polar modified polymer waxes include, but are not limited to, homopolymers and/or copolymers of C24, C25 and/or C26 groups, copolymers C26, C27 and/or C28 groups, or copolymers of C30-C38 groups, which have been modified with hydrophilic units such as, for example, maleic anhydride, acrylate, methacrylate, polyvinylpyrrolidone (PVP), etc. Preferably, the oil-soluble high carbon polar modified polymer wax has from about 5% to about 30% hydrophilic units, more preferably from about 10% to about 25% hydrophilic units by weight with respect to the weight of the wax, including all ranges and subranges therebetween. Particularly preferred hydrophilically modified waxes are C26, C27 and/or C28 homopolymers and copolymers which have been modified with maleic anhydride units.

Particularly preferred oil-soluble high carbon polar modified polymer waxes for use in the present invention are C26-C28 alpha olefin maleic acid anhydride copolymer waxes commercially available from Clariant under the trade name LICOCARE or LICOCENE. Specific examples of such waxes include products marketed by Clariant under the LicoCare name having designations such as CM 401, which is a maleic anhydride modified wax having a Mw of 2025 and a crystallinity of 11%, C30-C38 olefin/isopropylmaleate/maleic anhydride copolymer sold by Baker Hughes under the name Performa® V 1608, and C24-C26 alpha olefin acrylate copolymer wax commercially available from Clariant under the trade name LICOCARE CA301 LP3346 based on a polar backbone with C24-26 side chains with alternating ester and carboxylic acid groups.

According to other embodiments of the present invention, the polar modified polymer is not a wax. In accordance with these embodiments of the present invention, the polar modified polymer is based upon a homopolymer and/or copolymer of hydrophobic monomer(s) and has a weight-average molecular weight Mw of less than or equal to 1,000,000 g/mol, preferably of 1000 to 250,000 g/mol and particularly preferably of 5,000 to 50,000 g/mol, including all ranges and subranges therebetween.

In accordance with these embodiments, the polar modified polymer can be of any form typically associated with polymers such as, for example, block copolymer, a grafted copolymer or an alternating copolymer. For example, the polar modified polymer can contain a hydrophobic backbone (such as polypropylene and/or polyethylene) onto which hydrophilic groups (such as maleic anhydride) have been attached by any means including, for example, grafting. The attached groups can have any orientation (for example, atactic, isotactic or syndiotactic along the backbone).

Preferably, the oil-soluble high carbon polar modified polymer(s) represent from about 1% to about 20% of the total weight of the composition, more preferably from about 3% to about 17% of the total weight of the composition, and most preferably from about 5% to about 15%, including all ranges and subranges therebetween.

The oil soluble high carbon polar modified polymer is present in the composition of the invention in an amount ranging from about 2 to about 30% by weight, such as from about 3 to about 20% by weight, and from about 5 to about 10% by weight, including all ranges and subranges therebetween, all weights based on the total weight of the composition.

Reaction Product

According to preferred embodiments of the present invention, the oil-soluble polar modified polymer is reacted with the alkyl ethoxylated polymer wax, in the presence of oil to form a first reaction product. If the reaction is conducted at a relatively high temperature (for example, above 140° C.) and for a long period of time (>5 hours), a significant amount of the hydrophilic group (for example, carboxylic acid group associated with maleic anhydride groups) of the oil soluble polar modified polymer reacts with hydroxyl group(s) of the alkyl ethoxylated wax to yield a significant amount of the reaction product. If, however, the reaction is conducted at a relatively low temperature (for example, below 100° C.) and for a short period of time (<1 hour), only a small portion of the hydrophilic group of the polar modified polymer reacts with hydroxyl group(s)of the alkyl ethoxylated polymer wax to yield a minor amount of reaction product. Depending upon desired application, a minor amount or a significant amount of the first reaction product may be desired.

Water

The composition of the present invention further comprises water in order to form a water-in-oil/oil-in-water emulsion. The water is typically present in an amount of from about 30% to about 80% by weight, such as from about 40% to about 75% by weight, such as from about 50% to about 69% by weight, including all ranges and subranges therebetween, all weights based on the total weight of the composition.

High Molecular Weight Glycols

In some embodiments, the composition of the present invention can also include at least one high molecular weight glycol. Preferred high molecular weight glycols have a weight-average molecular weight of greater than about 1×10⁵ g/mol, preferably of greater than about 1×10⁶ g/mol, and most preferably of greater than about 2×10⁶ g/mol.

In accordance with the present invention, particularly preferred high molecular weight glycols are PEG-90M having a weight-average molecular weight of 4×10⁶ g/mol and PEG-45M having a weight-average molecular weight of 2.5×10⁶ g/mol, commercially are available from Amerchol under the trade names Polyox WSR-301 and Polyox WSR N-60K, respectively.

The high molecular weight glycol will typically be present in the composition of the invention in an amount of from about 0.01 to about 10.0% by weight, such as from about 0.05 to about 5% by weight, and all ranges therebetween, and from about 0.1 to about 2.0% by weight, including all ranges and subranges therebetween, all weights based on the weight of the composition.

Optional Ingredients

Non-Volatile Oil Capable of solubilizing the Polar Modified Polymers

The cosmetic compositions of the present invention can also optionally comprise at least one non-volatile oil capable of solubilizing the polar modified polymers. As used herein, the term “non-volatile” means having a boiling point of greater than about 100° C. The at least one non-volatile solvent typically comprises at least one non-volatile oil.

Examples of non-volatile oils that may be used in the present invention include, but are not limited to, polar oils such as:

hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;

synthetic oils or esters of formula R₅COOR₆ in which R₅ represents a linear or branched higher fatty acid residue containing from 1 to 40 carbon atoms, including from to 19 carbon atoms, and R₆ represents a branched hydrocarbon-based chain containing from 1 to 40 carbon atoms, including from 3 to 20 carbon atoms, with R₆ +R₇ 10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;

synthetic ethers containing from 10 to 40 carbon atoms;

C₈ to C₂₆ fatty alcohols, for instance oleyl alcohol; and

mixtures thereof.

The at least one non-volatile solvent for the oil soluble polar modified polymers is present in the cosmetic composition of the invention in an amount of from about 1% to about 50% by weight, such as from about 2% to about 40% by weight, such as from about 3% to about 30% by weight, all weights based on the total weight of the composition.

Volatile Solvent

The at least one volatile solvent may be chosen from a volatile silicone oil or a volatile non-silicone oil.

Suitable volatile silicone oils include, but are not limited to, linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | | Viscosity |
|---|---|---|
| Compound | Flash Point (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC | 56 | 1.5 |
| 200 (1.5 cSt) from Dow Corning | | |
| PDMS DC 200 (2 cSt) from Dow Corning | 87 | 2 |

Suitable volatile non-silicone oils may be selected from volatile hydrocarbon oils, alcohols, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate and their mixtures. Preferably, the volatile non-silicone oils have a flash point of at least 40° C.

Non-limiting examples of volatile non-silicone oils are listed in Table 2 below.

TABLE 2

| Compound | Flash Point (° C.) |
|---|---|
| Isododecane | 43 |
| Ethanol, denatured | 13 |
| Propylene glycol n-butyl ether | 60 |
| Ethyl 3-ethoxypropionate | 58 |
| Propylene glycol methylether acetate | 46 |
| Isopar L (isoparaffin C11-C13) | 62 |
| Isopar H (isoparaffin C11-C12) | 56 |

In general, the at least one volatile solvent is present in the composition in an amount of from about 1 to about 20% by weight, such as from about 2 to about 15% by weight, and from about 3 to about 10% by weight, all weights based on the total weight of the composition.

The composition of the present invention may also include any one, or more, optional ingredients. Examples thereof include, but are not limited to, colorants such as pigments and dyestuffs, co-solvents, plasticizers, preservatives, fillers, active ingredients, additional waxes and sunscreens.

The composition of the present invention is capable of creating a fiber on the end of a user's eyelash in the absence of any conventionally used fibers being dispersed in the composition.

It has surprisingly been discovered that the composition of the present invention forms a smudge proof, long wear emulsion, having a unique texture and feel, without the need for having to employ conventional latex or film forming polymers.

It has also surprisingly been discovered that the composition of the present invention forms a stable, elastic emulsion, that continuously provides clump free deposit. Without intending to be bound by theory, it is believed that the combination of the linear propylene-ethylene-maleic anhydride copolymer wax, C₂₆₋₂₈ α-olefin-maleic acid anhydride copolymer wax and alkyl ethoxylated polymer wax forms an interpolymer complex in an emulsion that can cause lengthening and allow the product to exhibit elasticity under tension force. In addition, the contribution of the crystallinity from the linear propylene-ethylene-maleic anhydride copolymer wax, C₂₆₋₂₈ α-olefin-maleic acid anhydride copolymer wax and alkyl ethoxylated polymer wax provides the hardening effects once deposited on to eye lashes. The hydrophilic portion of the alkyl ethoxylated polymer provides smudge proof properties and ease of removal from the eye lashes. Moreover, the composition of the present invention possesses a unique texture and feel, is long wearing and easily washable without the need for having to employ conventional latex or film forming polymers.

The rheological properties of the compositions in accordance with the present invention are determined by using a controlled stress rheometer, commercially available from TA Instruments under the name AR-G2. The samples are measured using a parallel plate having a stainless steel, cross hatched, 40 mm diameter plate. The gap is set at 1,000 microns. The desired temperature is precisely controlled by a Peltier system.

The mascara sample is transferred to the rheometer, and held at 25° C. for reaching temperature equilibrium. For a dynamic oscillation measurement, the sample is pre-sheared at a shear rate of 100 (1/second) for seconds, and followed by one minute at rest to reach equilibrium condition. The linear viscoelastic regime is determined in the oscillation strain sweep mode from 10⁻³% to 2×10³% of strain, at a constant frequency ω of 1 rad/s. The region at which the elastic modulus or storage modulus G′ is independent of % strain with an increasing oscillation strain is defined as a linear viscoelastic regime. The critical strain value is determined from the region at which the elastic modulus or storage modulus G′ begins to be dependent of oscillation strain. After reaching this critical strain, the internal material structure is disrupted and the mascara composition is no longer under linear viscoelastic region.

It is expected in conventional theory that when two dissimilar polar modified waxes are blended, an additive effect would be realized with respect to the storage modulus (G′) of the blend. Here, however, a surprising and unexpected synergy in storage modulus is achieved whereby the storage modulus (G′) for the blend is about four to about five times higher than the G′ of each individual polar modified wax. This phenomenon can be explained by the synergistic effect of the interpolymer complex formed by the blending of the two polar modified waxes.

The composition of the present invention is characterized by a storage modulus G′ ranging from about 100 Pa to about 10,000 Pa.

The yield % strain of the mascara composition refers to its viscoelastic properties when stress is applied. Typically, a high value of the yield % strain suggests more elastic property of the composition with a higher tolerance to stress before deforming into the non-linear viscoelastic regime. It is the yield % strain which characterizes the lengthening properties of the composition. It is unexpected and surprising to find that this invention can achieve a yield % strain that is about four to about five times more than the yield % strain of the individual polar modified waxes. This phenomenon can be explained by the synergistic effect of the interpolymer complex formed by the blending of the two polar modified waxes.

The shear viscosity η(\dot{γ}) of the mascara composition is measured in the flow mode. Before measuring the shear viscosity, the sample is pre-sheared at a shear rate \dot{γ} of 100 (1/second) for 30 seconds, and allowed to rest for one minute to reach equilibrium condition. Then, viscosity of the sample is measured in the continuous ramp mode from 10⁻³ (1/second) to 10² (1/second) for 10 minutes. A high value of the shear viscosity η(\dot{γ}) at low shear rate indicates high stability of structure at rest which is highly essential for storage stability.

The degree of shear thinning can be indicative of the ease of application and the deposit of product on the substrate, especially for eye lashes. This value is determined from the slope of log-log curve of shear viscosity η(\dot{γ}) versus shear rate \dot{γ} which should be in the range of from about −0.6 to less than −1.0 for good shear thinning behaviors. It has been found that the composition of the present invention possesses a desirable high degree of shear thinning of −0.91.

It has also been surprisingly discovered that the composition of the present invention is able to enhance lengthening extensions on eye lashes. More particularly, the present invention can produce evenly visible extension onto the eye lashes upon applying the mascara composition while holding the applicator at a set distance until solvents evaporate. The said mascara composition can produce extensions up to 10 mm, most preferably up to 4 mm evenly on false eye lashes.

The present invention is further described in terms of the following non-limiting examples. Unless otherwise indicated, all parts and percentages are on a weight-by-weight percentage basis.

### EXAMPLES OF LENGTHENING MASCARAS

### Examples 1-3

| | | Comparative | Comparative | Inventive |
|---|---|---|---|---|
| | | Example 1 | Example 2 | Example 3 |
| Phase | INCI | Wt/wt % | Wt/wt % | Wt/wt % |
| A | C20-C40 Pareth-10 | 10 | 10 | 10 |
| A | C26-C28 ALPHA | 10 | 0 | 5 |
| | OLEFIN MALEIC ACID | | | |
| | ANHYDRIDE | | | |
| | COPOLYMER | | | |
| A | Propylene-ethylene- | 0 | 10 | 5 |
| | MALEIC ANHYDRIDE | | | |
| | COPOLYMER | | | |
| A | Iron Oxides | 5 | 5 | 5 |
| B | DI Water | 63.25 | 63.25 | 63.25 |
| B | Pentylene Glycol | 2 | 2 | 2 |
| B | PVP | 7.5 | 7.5 | 7.5 |
| B | NaOH | 0.5 | 0.5 | 0.5 |
| C | Simethicone | 0.1 | 0.1 | 0.1 |
| D | Preservatives | 1.65 | 1.65 | 1.65 |
| | Total | 100 | 100 | 100 |

Procedure
1. In main tank A, the following were added: C20-C40 Paret-10, C₂₆₋₂₈ α-olefin-maleic acid anhydride copolymer and Propylene-ethylene-maleic Anhydride copolymer. The contents were then heated to 90° C. until all solids had melted.
2. When all solids had melted, contents were homogenized while Iron Oxides were being added to the batch. Batch was homogenized for at least 1 hr at 900 rpm.
3. In side tank B with water bath, DI water, PVP, NaOH, Pentylene Glycol were added and mixed until homogeneous. The contents were heated to 85° C. with moderate agitation.
4. When both tanks were at 85° C., side tank B was slowly added to main tank A with a water bath while homogenizing at 900 rpm.
5. After 5 minutes of homogenizing, Simethicone was added to the batch. Batch was homogenized for an additional 30 minutes at 85° C. with 900 rpm.
6. Batch was cooled to 25° C. with a planetarium blade.
7. At 35° C., preservatives were added and mixed until uniform.
8. Batch was poured to container at 25° C.

### Example 4

### Lengthening Mascara

| | | Example 4 |
|---|---|---|
| Phase | INCI | Wt/wt % |
| A | C20-C40 Pareth-10 | 10 |
| A | C26-C28 ALPHA OLEFIN MALEIC ACID | 7 |
| | ANHYDRIDE COPOLYMER | 7 |
| A | Propylene-ethylene-MALEIC | 3 |
| | ANHYDRIDE COPOLYMER | |
| A | Iron Oxides | 5 |
| B | DI Water | Q.S. |
| B | PVP | 5 |
| B | NaOH | 0.75 |
| B | Pentylene Glycol | 6 |
| B | PEG-90M | 0.3 |
| C | Simethicone | 0.1 |
| D | Preservatives | 1.65 |
| | Total | 100 |

### Procedure

1. In main tank A, the following were added: C20-C40 Paret-10, C₂₆₋₂₈ α-Olefin Maleic Acid Anhydride Copolymer and Propylene-ethylene-Maleic Anhydride copolymer. The contents were then heated to 90° C. until all solids had melted.
2. When all solids had melted, contents were homogenized while Iron Oxides were being added to the batch. Batch was homogenized for at least 1 hr at 900 rpm.
3. In side tank B with water bath, DI water, PVP, NaOH, Pentylene Glycol, PEG-90M were added and mixed until homogeneous. The contents were heated to 85° C. with moderate agitation.
4. When both tanks were at 85° C., side tank B was slowly added to main tank A with a water bath while homogenizing at 900 rpm.
5. After 5 minutes of homogenizing, Simethicone was added to the batch. Batch was homogenized for an additional 30 minutes at 85° C. with 900 rpm.
6. Batch was cooled to 25° C. with a planetarium blade.
7. At 35° C., preservatives and alcohol were added and mixed until uniform.
8. Batch was poured to container at 25° C.

### Rheological Values for Examples 1-4

| | G′ at 0.1% | | Viscosity at | Shear |
|---|---|---|---|---|
| | strain | Yield % | 0.1 1/s | Thinning |
| Examples | (Pa) | strain | (Pa · s) | Slope |
| 1 | 906.5 | 0.8 | 644.6 | −0.91 |
| 2 | 731.5 | 1.0 | 1225 | −0.99 |
| 3 | 4254 | 4.0 | 4114 | −0.93 |
| 4 | 8425 | 4.2 | 1492.5 | −0.97 |

As is shown by the above data, inventive Example 3 experienced synergistic rheological effects when the two polar modified polymers were combined in said composition, such as a four to five times higher value of G′. Furthermore, inventive Example 3 had a much higher yield % strain which correlates to the lengthening effect of this composition.

### Example 5—Comparative Example

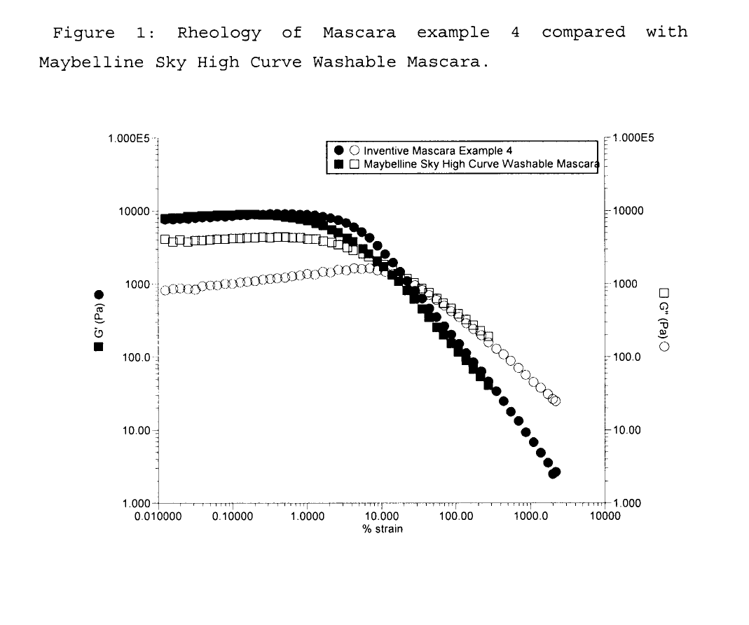

The Mascara of Example 4 was compared to commercially available Maybelline Sky High Curve Washable Mascara. In comparing Sky High Curve with Example 4, Example 4 exhibits at least double the yield strain than that of Sky High Curve. Therefore, it can be seen that inventive mascara Example 4 has greater lengthening than Sky High Curve. Morever, the shear thinning behavior was not compromised with a higher shear viscosity as shown by the shear thinning slope of −0.93, indicating that the composition had good application and deposit properties. (See, FIG. 1).

What is claimed is:

1. A composition comprising:
(a) at least one alkyl ethoxylated polymer wax;
(b) at least one oil soluble polar modified polymer;
(c) at least one oil soluble high carbon polar modified polymer;
(d) water;
(e) optionally, at least one non-volatile oil capable of solubilizing the polar modified polymers;
(f) optionally, at least one volatile solvent; and
(g) optionally, at least one colorant, and wherein the composition does not require fibers to be dispersed therein.

2. The composition of claim 1 wherein (a) is a Pareth-10 alcohol.

3. The composition of claim 1 wherein (a) is present in an amount of from about 3 to about 30% by weight, based on the weight of the composition.

4. The composition of claim 1 wherein (b) is present in an amount of from about 2 to about 30% by weight, based on the weight of the composition.

5. The composition of claim 1 wherein (c) is present in an amount of from about 2 to about 30% by weight, based on the weight of the composition.

6. The composition of claim 1 wherein (d) is present in an amount of from about 30 to about 80% by weight, based on the weight of the composition.

7. The composition of claim 1 wherein (e) is present in an amount of from about 1 to about 50% by weight, based on the weight of the composition.

8. The composition of claim 1 wherein (f) is present in an amount of from about 1 to about 20% by weight, based on the weight of the composition.

9. A method of lengthening eyelashes comprising applying onto the eyelashes a composition comprising:
(a) at least one alkyl ethoxylated polymer wax;
(b) at least one oil soluble polar modified polymer;
(c) at least one oil soluble high carbon polar modified polymer;
(d) water;
(e) optionally, at least one non-volatile oil capable of solubilizing the polar modified polymers;
(f) optionally, at least one volatile solvent; and
(g) optionally, at least one colorant, and wherein the composition does not require fibers to be dispersed therein.

10. A composition comprising:
(a) at least one alkyl ethoxylated polymer wax;
(b) at least one oil soluble polar modified polymer;
(c) at least one oil soluble high carbon polar modified polymer;
(d) water;
(e) optionally, at least one non-volatile oil capable of solubilizing the polar modified polymers;
(f) optionally, at least one volatile solvent;
(g) at least one high molecular weight glycol; and
(h) optionally, at least one colorant, and wherein the composition does not require fibers to be dispersed therein.

11. The composition of claim 10 wherein (a) is a Pareth-10 alcohol.

12. The composition of claim 10 wherein (a) is present in an amount of from about 3 to about 30% by weight, based on the weight of the composition.

13. The composition of claim 10 wherein (b) is present in an amount of from about 2 to about 30% by weight, based on the weight of the composition.

14. The composition of claim 10 wherein (c) is present in an amount of from about 2 to about 30% by weight, based on the weight of the composition.

15. The composition of claim 10 wherein (d) is present in an amount of from about 30 to about 80% by weight, based on the weight of the composition.

16. The composition of claim 10 wherein (e) is present in an amount of from about 1 to about 50% by weight, based on the weight of the composition.

17. The composition of claim 10 wherein (f) is present in an amount of from about 1 to about 20% by weight, based on the weight of the composition.

18. The composition of claim 10 wherein (g) is present in an amount of from about 0.01 to about 10.0% by weight, based on the weight of the composition.

19. A method of lengthening eyelashes comprising applying onto the eyelashes a composition containing:
(a) at least one alkyl ethoxylated polymer wax;
(b) at least one oil soluble polar modified polymer;
(c) at least one oil soluble high carbon polar modified polymer;
(d) water;
(e) at least one non-volatile oil capable of solubilizing the polar modified polymers;
(f) optionally, at least one volatile solvent;
(g) at least one high molecular weight glycol; and
(h) optionally, at least one colorant, and wherein the composition does not require fibers to be dispersed therein.
